(19) United States
(12) Patent Application Publication
(10) Pub. No.: US 2024/0136030 A1
(43) Pub. Date: Apr. 25, 2024

(54) PATIENT-REPORTED ASSESSMENT TOOL DEDICATED FOR ASSESSMENT OF OXALIPLATIN-INDUCED PERIPHERAL NEUROPATHY (OIPN)

(71) Applicant: Jiangsu Province Academy of Traditional Chinese Medicine, Nanjing (CN)

(72) Inventors: Zhancheng GU, Nanjing (CN); Jiege HUO, Nanjing (CN); Guoli WEI, Nanjing (CN)

(21) Appl. No.: 18/530,425

(22) Filed: Dec. 6, 2023

(30) Foreign Application Priority Data
Oct. 31, 2023 (CN) 202311448177.3

(51) Int. Cl. G16H 10/20; G16H 50/30

(52) U.S. Cl. CPC G16H 10/20; G16H 50/30

(57) ABSTRACT

The present invention discloses a patient-reported assessment tool dedicated for assessment of oxaliplatin-induced peripheral neuropathy (OIPN), comprising a main scale, a subscale and a scoring formula. The assessment contents of the main scale contain symptoms of body organs and are graded based on patient's subjective feelings about the frequency of the neuropathy, corresponding to relative scores. The assessment contents of the subscale contain location, duration and influence of the most severe symptom. The present invention effectively improves the accuracy and sensitivity of CIPN assessment through the targeted application range design and the maximum item weight addition, and improves the clinical reference value of CIPN assessment by elaborating the description of OIPN symptoms and integrating the features of several validated assessment tools, and provides reference for the development of CIPN assessment tools.

### TECHNICAL FIELD

The present invention relates to the technical field of medical assessment tools, and particularly relates to a patient-reported assessment tool dedicated for assessment of oxaliplatin-induced peripheral neuropathy (OIPN).

### BACKGROUND

Chemotherapy-induced peripheral neuropathy (CIPN) is a common side effect during antitumor therapy, and is the main cause for reduction or termination of chemotherapy. Among which, OIPN is the mostly concerned. The development of CIPN prevention and control strategies is not optimistic, which is not only because of the complexity of the underlying molecular mechanisms of different chemotherapy drugs, but also more importantly because of the lack of a sensitive and targeted assessment tool to monitor the curative effect of prevention and control of CIPN. At present, questionnaires based on patient-reported outcome have become the most commonly used CIPN assessment tools, such as the Functional Assessment of Cancer Therapy/Gynecologic Oncology Group-Neurotoxicity (FACT/GOG-Ntx) questionnaire, the European Organization for Research and Treatment of Cancer Quality of Life Questionnaire—Chemotherapy-Induced Peripheral Neuropathy 20 Items (EORTC QLQ-CIPN20), the Patient Neurotoxicity Questionnaire (PNQ), the Chemotherapy-Induced Peripheral Neuropathy Assessment Tool (CIPNAT), the Comprehensive Assessment Scale for Chemotherapy-Induced Peripheral Neuropathy (CAS-CIPN) and the Treatment-Induced Neuropathy Assessment Scale (TNAS). However, due to the pursuit of universality, some specific symptoms are often ignored in such tools, resulting in a low overall positive response rate and a floor effect (the total scores are low and similar). The floor effect will cause a low sensitivity and poor interference immunity of the assessment results, which could make the results more likely to be affected by other basic neurological diseases and produce biases. Therefore, the assessment results are difficult to reflect the differences in the severity of neuropathy in the scores, and thus are difficult to provide a substantive guidance for intervention measures.

### SUMMARY

### (I) Purpose of the Present Invention

In view of this, the purpose of the present invention is to provide a patient-reported assessment tool dedicated for assessment of oxaliplatin-induced peripheral neuropathy (OIPN), so as to enhance the reliability and clinical practicability of CIPN assessment and provide a technical support for the prevention and control strategies of CIPN.

### (II) Technical Solution

The present invention is a patient-reported assessment tool dedicated for assessment of OIPN, named chemotherapy-induced peripheral neuropathy integrated assessment—oxaliplatin subscale (CIPNIA-OS), comprising a main scale, a subscale and a scoring formula. The assessment contents of the main scale contain symptoms of body organs and are graded based on patient's subjective feelings about the frequency of the neuropathy, corresponding to relative scores. The assessment contents of the subscale contain location, duration and influence of the most severe symptom.

Preferably, the symptoms of body organs include a total of 17 items: tingling, numbness, and oversensitivity or pain of hands or fingers; tingling, numbness, and oversensitivity or pain of feet or toes; tingling and numbness around mouth; cramps and weakness of hands or arms; cramps and weakness of feet or legs; contraction sense in throat; difficulty in writing or buttoning; difficulty in holding or grabbing; difficulty in standing balance; and difficulty in walking or ascending steps.

Preferably, the grades include 4 levels: not at all, a little bit, quite a bit, very much, corresponding to the scores of 0, 1, 2 and 3, respectively.

Preferably, the scores include 4 grades: 0, 1, 2 and 3, corresponding to the 4 levels of grades, respectively.

Preferably, the location of the most severe symptom is divided into 4 grades: nowhere, limited to fingers or toes, extending to wrist or ankle, above wrist or ankle, corresponding to the scores of 0, 1, 2 and 3, respectively.

Preferably, the duration of the most severe symptom is divided into 4 grades: 0, ≤1 week, ≤3 weeks, and >3 weeks, corresponding to the scores of 0, 1, 2 and 3, respectively.

Preferably, the influence of the most severe symptom is divided into 4 grades: no, mild, limited tool-using, and limited self-care, corresponding to the scores of 0, 1, 2 and 3, respectively.

Preferably, the scoring formula is S=T₁−tₘₐₓ+tₘₐₓ×T₂.

It can be seen from the above technical solution that the present application has the following beneficial effects:

1. The present invention effectively improves the accuracy and sensitivity of CIPN assessment through the targeted application range design and the maximum item weight addition.

2. The present invention effectively improves the clinical reference value of CIPN assessment by elaborating the description of OIPN symptoms and integrating the features of several validated assessment tools, and provides reference for the development of CIPN assessment tools.

### DESCRIPTION OF DRAWINGS

FIG. 1 is a schematic diagram of a main scale of the present invention;

FIG. 2 is a schematic diagram of a subscale of the present invention;

FIG. 3 is a schematic diagram of reliability analysis of a main scale of the present invention;

FIG. 4 is a schematic diagram of structural validity analysis of a main scale of the present invention;

FIG. 5 is a schematic diagram of exploratory factor analysis of a main scale of the present invention.

### DETAILED DESCRIPTION

The description below is illustrative in nature and is not intended to limit the present disclosure, application and use. It should be understood that in all the drawings, same or similar reference signs refer to same or similar parts and features. The drawings only schematically show the conception and principle of the embodiments of the present disclosure, but do not necessarily show the specific dimensions and proportions of the embodiments of the present disclosure. Relevant details or structures of the embodiments of the present disclosure may be illustrated in a specific portion of a specific drawing in an exaggerated manner.

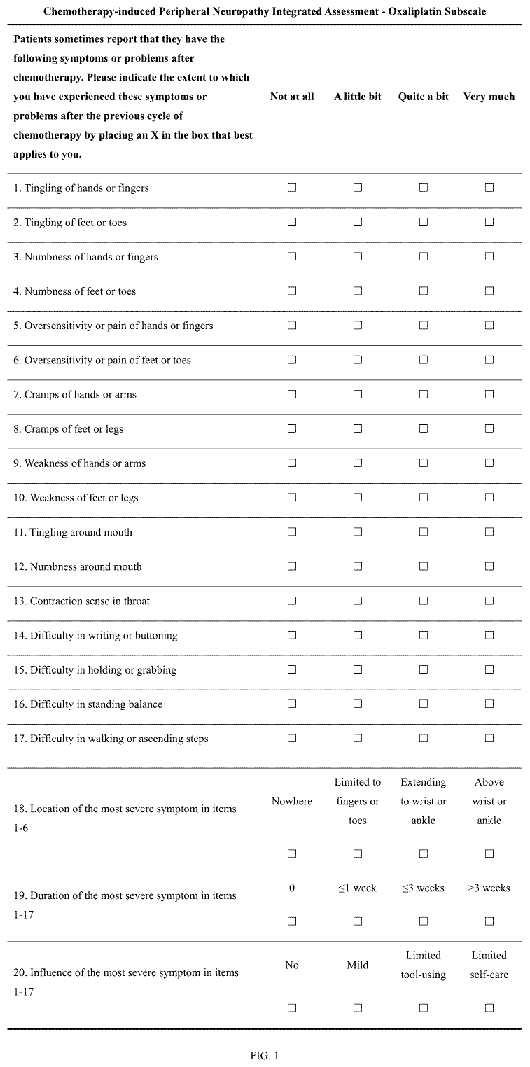

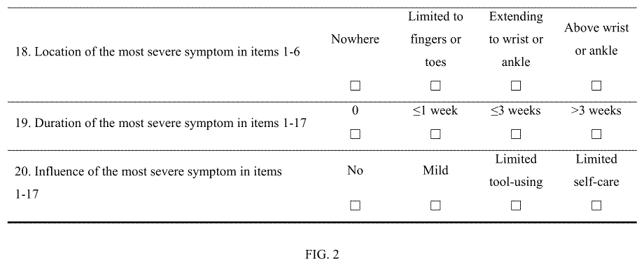

Referring to FIGS. 1-5, an embodiment provided by the present invention is:

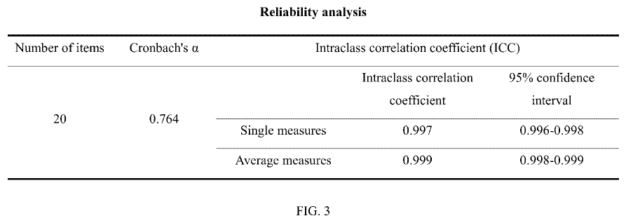

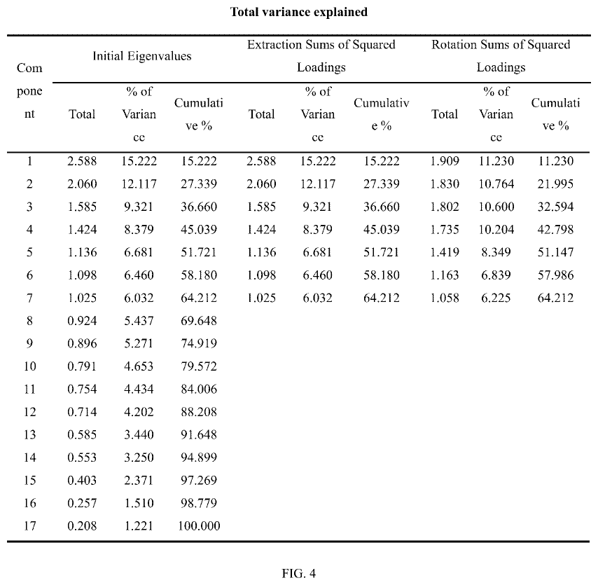

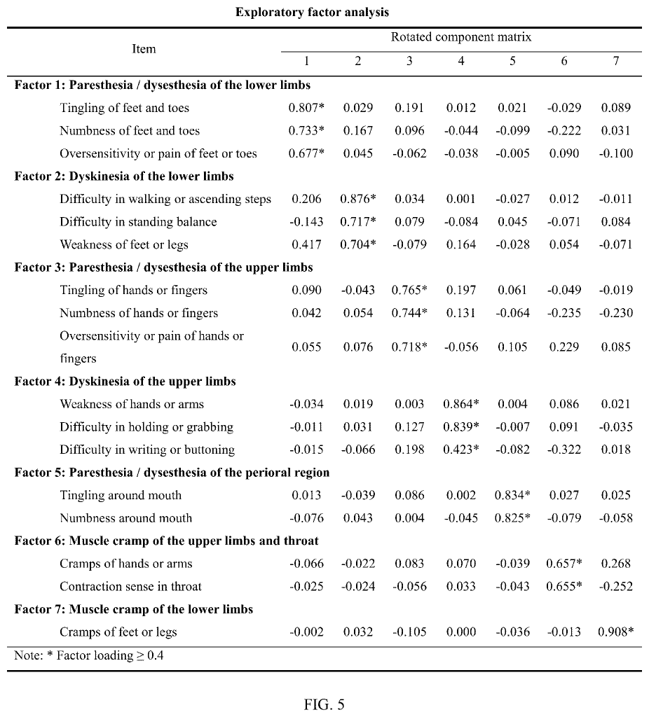

The present invention is a patient-reported assessment tool dedicated for assessment of oxaliplatin-induced peripheral neuropathy (OIPN), comprising a main scale, a subscale and a scoring formula. The assessment contents of the main scale contain symptoms of body organs and are graded based on patient's subjective feelings about the frequency of the neuropathy, corresponding to relative scores. The assessment contents of the subscale contain location, duration and influence of the most severe symptom. Cronbach's a is used as the index of the reliability of the main scale, and the results are shown in FIG. 3. The Cronbach's a of the whole CIPNIA-OS is 0.764 (greater than 0.7), indicating that the internal consistency of the 20 items in the whole questionnaire is considerable. The intraclass correlation coefficient (ICC) from the test-retest reliability is 0.997, which is between 0.9 and 1, indicating that the stability of CIPNIA-OS is good. The structure of the main scale is analyzed by exploratory factor analysis. The KMO coefficient is 0.694 (>0.5), indicating there is strong correlation between variables. Bartlett's test of sphericity indicates that each variable is independent and the correlation matrix is factorable (χ²=5726.739, p<0.05). Factor loading of the rotated component matrix is shown in Table 3. Factor loadings ≥0.40 are marked to explore item commonalities, and factor explanations are provided in FIG. 5. A total of 7 common factors are extracted by principal component analysis, which are basically distributed according to upper limb, lower limb and perioral symptoms. The cumulative variance explanation rate of the factors is 64.21% (>50%), as shown in FIG. 4, indicating that the structural validity of the questionnaire is acceptable.

Further, the symptoms of body organs include a total of 17 items: tingling, numbness, and oversensitivity or pain of hands or fingers; tingling, numbness, and oversensitivity or pain of feet or toes; tingling and numbness around mouth; cramps and weakness of hands or arms; cramps and weakness of feet or legs; contraction sense in throat; difficulty in writing or buttoning; difficulty in holding or grabbing; difficulty in standing balance; and difficulty in walking or ascending steps. Such detailed description helps the medical professional gain a more complete understanding of a patient's symptoms, thus to make the diagnosis and treatment plan more accurately.

Further, the grades include 4 levels: not at all, a little bit, quite a bit, very much. Grading according to the patients' cognition of symptom occurrence frequency can more accurately reflect the subjective perceived symptoms of neuropathy, which is closer to the real situation than reported by clinicians.

Further, the scores include 4 grades: 0, 1, 2 and 3, corresponding to the 4 levels of grades, respectively. The scores are introduced to assess and record the severity of the symptoms, therefore more accurate quantitative information is provided, which allows a medical professional to quantify and grasp the evaluation results more intuitively, thus to make a more targeted treatment plan and measure the curative effect more accurately.

Further, the location of the most severe symptom is divided into 4 grades: nowhere, limited to fingers or toes, extending to wrist or ankle, above wrist or ankle, corresponding to the scores of 0, 1, 2 and 3, respectively. Such division helps a medical professional understand the location and spread of the symptoms more accurately, thus to grasp the severity of symptoms better and develop a more appropriate treatment strategy.

Further, the duration of the most severe symptom is divided into 4 grades: 0, ≤1 week, ≤3 weeks, and >3 weeks. Such division helps a medical professional determine the time frame for the onset of symptoms, thus to grasp the severity of symptoms better and develop a more appropriate treatment strategy

Further, the influence of the most severe symptom is divided into 4 grades: no, mild, limited tool-using, and limited self-care. Such division helps a medical professional quantify the impact on the quality of a patient's life and gain a more complete understanding of the patient's vital function and limitation extent in daily activities, thus to grasp the severity of symptoms better and develop a more appropriate treatment strategy.

Further, the scoring formula is S=T₁−tₘₐₓ+tₘₐₓ×T₂, wherein S is an overall score, T₁ is a total score of the main scale, tₘₐₓ is the highest individual score of the main scale, and T₂ is a total score of the subscale.

For those skilled in the art, apparently, the present invention is not limited to details of the above demonstrative embodiments. Moreover, the present invention can be realized in other specific forms without departing from the spirit or basic feature of the present invention. Therefore, in all respects, the embodiments shall be regarded to be demonstrative and nonrestrictive. The scope of the present invention is defined by appended claims, rather than the above description. Therefore, the present invention is intended to include all changes falling into the meaning and the scope of equivalent elements of claims within the present invention. Any drawing mark in claims shall not be regarded to limit the concerned claims.

1. A patient-reported assessment tool dedicated for assessment of oxaliplatin-induced peripheral neuropathy (OIPN), comprising a main scale, a subscale and a scoring formula, wherein the assessment contents of the main scale contain symptoms of body organs and are graded based on patient's subjective feelings about the frequency of the neuropathy, corresponding to relative scores, and the assessment contents of the subscale contain location, duration and influence of the most severe symptom.

2. The patient-reported assessment tool dedicated for assessment of oxaliplatin-induced peripheral neuropathy (OIPN) according to claim 1, wherein the symptoms of body organs include a total of 17 items: tingling, numbness, and oversensitivity or pain of hands or fingers; tingling, numbness, and oversensitivity or pain of feet or toes; tingling and numbness around mouth; cramps and weakness of hands or arms; cramps and weakness of feet or legs; contraction sense in throat; difficulty in writing or buttoning; difficulty in holding or grabbing; difficulty in standing balance; and difficulty in walking or ascending steps.

3. The patient-reported assessment tool dedicated for assessment of oxaliplatin-induced peripheral neuropathy (OIPN) according to claim 1, wherein the grades include 4 levels: not at all, a little bit, quite a bit, very much.

4. The patient-reported assessment tool dedicated for assessment of oxaliplatin-induced peripheral neuropathy (OIPN) according to claim 1, wherein the scores include 0, 1, 2 and 3.

5. The patient-reported assessment tool dedicated for assessment of oxaliplatin-induced peripheral neuropathy (OIPN) according to claim 1, wherein the location of the most severe symptom is divided into 4 grades: nowhere, limited to fingers or toes, extending to wrist or ankle, above wrist or ankle.

6. The patient-reported assessment tool dedicated for assessment of oxaliplatin-induced peripheral neuropathy (OIPN) according to claim 1, wherein the duration is divided into 4 grades: 0, ≤1 week, ≤3 weeks, and >3 weeks.

7. The patient-reported assessment tool dedicated for assessment of oxaliplatin-induced peripheral neuropathy (OIPN) according to claim 1, wherein the influence of the most severe symptom is divided into 4 grades: no, mild, limited tool-using, and limited self-care.

8. The patient-reported assessment tool dedicated for assessment of oxaliplatin-induced peripheral neuropathy (OIPN) according to claim 1, wherein the scoring formula is S=T₁−tₘₐₓ+tₘₐₓ×T₂.
